(19) United States
(12) Patent Application Publication
(10) Pub. No.: US 2025/0248623 A1
(43) Pub. Date: Aug. 7, 2025

(54) INSERTER ASSEMBLY AND METHOD

(71) Applicant: Unomedical A/S, Lejre (DK)

(72) Inventors: Matthias Heschel, Lejre (DK); Steffen Gyrn, Lejre (DK)

(21) Appl. No.: 19/045,075

(22) Filed: Feb. 4, 2025

Related U.S. Application Data
(63) Continuation of application No. PCT/EP2023/071746, filed on Aug. 4, 2023.
(60) Provisional application No. 63/395,472, filed on Aug. 5, 2022.

(30) Foreign Application Priority Data
Aug. 18, 2022 (GB) 2212059.6

(51) Int. Cl. A61B 5/145; A61B 5/00

(52) U.S. Cl. CPC A61B 5/14503; A61B 5/14532; A61B 5/6848; A61B 2560/0214; A61B 2560/0462; A61B 2560/06; A61B 2562/227

(57) ABSTRACT

Certain embodiments relate to an inserter assembly for inserting an analyte sensing device into subcutaneous tissue of a body. In certain embodiments, the inserter assembly generally includes an insertion device, a sensor carrier, a sensor assembly, and a sensor base. The insertion device includes a cover with a first end and an opposite second end. The sensor carrier is placed inside the cover, and includes an injection means. The sensor carrier is operable to be moved from a first position to a second position by activating a first energy storage unit, which is activated by a deployment device placed in the insertion device. The sensor assembly includes the analyte sensing device, and is adapted to be inserted and placed into the subcutaneous tissue when the sensor carrier is in the second position. The sensor base is placed at the second end of the cover, and includes an adhesive.

### TECHNICAL FIELD

The present disclosure relates to inserter assemblies, and more particularly but not exclusively relates to inserter assemblies for inserting an analyte sensing device such as a continuous glucose monitoring sensor into a subcutaneous tissue of a body.

### BACKGROUND

US Publication No. 2012/0226122 discloses an inserter device for an analyte sensor. The device includes a housing comprising a blade shuttle, and a sensor shuttle. A spring is compressed between the blade shuttle and the sensor shuttle. The blade shuttle and sensor shuttle move towards the subcutaneous layer and when the spring force is released, the blade shuttle moves towards the subcutaneous layer and pierces it, thereby making a pathway into the subcutaneous layer. The movement of the sensor shuttle implants the analyte sensor. Then, the blade shuttle is retracted from the subcutaneous fat layer and leaving the analyte sensor in the subcutaneous layer.

EP2691144, US2019/0001055 and U.S. Pat. No. 10,076,607B2 disclose various other inserter devices.

### SUMMARY

Certain embodiments of the subject matter disclosed herein seek generally to provide improved inserter assemblies for inserting an analyte sensing device (such as a continuous glucose monitoring sensor). Certain embodiments may overcome one or more of the abovementioned deficiencies and drawbacks of conventional inserter assemblies and/or provide a useful alternative to existing options.

The invention is defined in the appended claims.

According to a first aspect, an inserter assembly for inserting an analyte sensing device into subcutaneous tissue of a body is disclosed. The inserter assembly comprises:
an insertion device comprising a cover with a first end and an opposite second end; and
a sensor carrier placed inside the cover, the sensor carrier comprising an injection means, wherein the sensor carrier is operable to be moved from a first position to a second position by activating a first energy storage unit, wherein the first energy storage unit is activated by a deployment device placed in the insertion device;
a sensor assembly comprising an analyte sensing device, wherein the sensor assembly is adapted to be inserted and placed into the subcutaneous tissue when the sensor carrier is in the second position;
a sensor base placed at the second end of the cover, the sensor base comprising a first surface and an opposite second surface, the second surface comprising an adhesive;
wherein the sensor assembly comprises a sensor module connected to the analyte sensing device, wherein the sensor module is adapted to be moved together with the analyte sensing device by a piston;
wherein, with the sensor carrier in the second position, the sensor module is adapted to be attached to the first surface of the sensor base; and
wherein the analyte sensing device is placed in the subcutaneous tissue when the sensor carrier reaches its second position.

With this arrangement, the sensor module becomes attached to the sensor base in the second position. In the first position, the sensor module is detached (spaced from) from the sensor base.

In moving from the first position to the second position, the sensor module moves towards the second end of the cover, i.e. towards the skin.

Aptly, the inserter assembly further comprises a second energy storage unit;
wherein the second energy storage unit is activated by activating means when the sensor carrier reaches its second position; wherein the sensor carrier is adapted to be moved, by the activating means, to a third position different from the second position, and in a direction towards the first end of the cover.

Aptly, the first energy storage unit comprises a first spring configured to bring the sensor carrier from the first position to the second position; and wherein the second energy storage unit comprises a second spring adapted to transport the sensor carrier to the third position; and wherein a longitudinal central axis of the first spring is coincident with a longitudinal central axis of the second spring.

Aptly, the first spring is a coil spring placed inside the piston and surrounding the sensor carrier; wherein, when the sensor carrier is in the first position, the first spring is compressed, with a first free end placed nearest the second end of the cover and pressing towards an inside surface of the piston, and the second opposite free end of the first spring is pressed towards a part of the inserter assembly placed opposite the inside surface of the piston.

Aptly, the second spring is a coil spring and placed inside the sensor carrier in a cavity; wherein a first free end of the second spring abuts an internal base part of the piston; and wherein an opposite second free end of the second spring abuts an internal upper surface of the sensor carrier.

Aptly, the sensor base is adapted to cover a passage encircled by the second end of the cover, whereby the sensor carrier and the sensor assembly are enclosed in a cavity formed by the cover and the sensor base; and wherein the inserter assembly comprises means adapted to attach the sensor base to the insertion device.

Aptly, an inner housing encircles the piston, and a base lock is placed around the inner housing; wherein the base lock is slidable in a longitudinal direction of the inserter assembly; wherein the inner housing comprises releasable locking means adapted to lock the sensor base to the insertion device when the sensor carrier is in the first position; and wherein the locking means is adapted to be released by the base lock when the sensor carrier is in the second position to thereby release the sensor base from the insertion device.

Aptly, the base lock comprises a sleeve surrounding a portion of the inner housing; and wherein the inserter assembly further comprises flexible arms, longitudinal axes of which are parallel with a longitudinal axis of the inserter assembly.

Aptly, the sensor module comprises electrical connectors for connecting to an electrical circuitry and for transmitting signals received from the analyte sensing device to a transmitter. The transmitter may be attachable to the sensor base after the sensor base has been attached to the skin and the insertion device removed.

Aptly, the transmitter comprises electrical circuitry for transmitting signals received from the sensor assembly to a remote unit; and wherein the transmitter further comprises a power supply.

Aptly the first surface of the sensor base comprises a first area adapted to receive the sensor module; wherein the sensor base further comprises a second area separated from the first area, the second area comprising a through-going bore in said sensor base; wherein said through-going bore is adapted to receive a part of the analyte sensing device and to secure an upper part of the analyte sensing device to the sensor base.

Aptly, the second area further is adapted to fasten an upper part of the analyte sensing device to the sensor base while a lower part of the analyte sensing device comprises an elongated body that is injected through the bore and is placed in the subcutaneous tissue when the sensor carrier reaches its second position.

Aptly, the injection means comprises an insertion needle, which in sectional view is C-shaped and comprises a sharp tip and a lateral opening delimited by sidewalls, and wherein the lateral opening comprises a sharp region at each of the sidewalls.

Aptly, the sensor carrier comprises a needle hub attaching the insertion needle; and wherein the needle hub is configured to be moved by the piston from the first to the second position.

Aptly, the sensor carrier comprises a hollow part in which the needle hub is placed; and wherein the second energy storage unit is placed in a circular cavity arranged between the outside of the needle hub and the inside of the hollow part when the sensor carrier is in the first position and the second position.

Aptly the piston comprises a lower piston surface part adapted to abut against a top surface of the sensor module, wherein said piston surface and the injection needle are moved synchronously from the first position to the second position, and the injection needle and the lower piston surface part are placed in a distance from each other.

Aptly the assembly further comprises flexible elongated retention arms by which the sensor module is operable to be attached to the first surface of the sensor base, the retention arms comprising retention means adapted to be locked into recesses.

Aptly the activating means is configured to cause a rotating movement of the sensor carrier whereby locking of the sensor carrier to the piston is released, and a force of the second energy storage unit is released thereby moving the sensor carrier from the second position to the third position.

Aptly the inserter assembly comprises a release part comprising a cap with a cap surface placed between the deployment device and the inner housing, and a surrounding cap-portion encircling an upper part of the outer surface of the inner housing, and further comprising flexible arms locking the deployment device when the sensor carrier is in the first position.

Aptly the deployment device comprises a pressure unit comprising a first surface to which pressure is applied and a second opposite surface region adapted to lock flexible arms placed in the release part towards a lower edge of the deployment device; wherein the flexible arms comprise retention units; and wherein by a pressure applied to the first surface the retention units are moved to thereby release the piston, whereby a force stored in the first energy storing device is released and moves the sensor carrier from the first position to the second position.

Aptly the piston comprises locking means resting on an upper edge of the release part; wherein said locking means are adapted to be released from the release part by a lateral movement of the upper part, which takes place when the retention units are moved.

Aptly, the first energy storage unit is preloaded with a first expansion force; wherein and the second energy storage unit is preloaded with a second expansion force; and wherein the first expansion force is larger than the second expansion force.

Aptly the assembly further comprises a piston assembly comprising the release part, a first energy storing device, an insertion needle fastened to a needle hub, a second energy storing device, and the piston.

According to another aspect there is provided an inserter assembly for inserting an analyte sensing device into subcutaneous tissue of a body, said inserter assembly comprising:
an insertion device comprising a cover with a first end and an opposite second end and a sensor carrier placed inside the cover, wherein said sensor carrier is moved from a first position to a second position by activating a first energy storage unit, wherein the first energy storage unit is activated by a deployment device placed in the insertion device;
a sensor assembly comprising:
   the analyte sensing device, wherein the sensor assembly adapted to be inserted and placed into the tissue when the sensor carrier is in the second position;
   a sensor base placed at the second end of the cover, the sensor base comprising a first surface and an opposite second surface, the second surface comprising an adhesive; and
   a sensor module connected to the analyte sensing device, said sensor module is adapted to be moved together with the analyte sensing device by a piston;
wherein, with the sensor carrier in the second position, the sensor module is adapted to be attached to the first surface of the sensor base; and
wherein the analyte sensing device is by injection means placed in the subcutaneous tissue when the sensor carrier reaches its second position.

Aptly the injection means comprises a sharp tip of the analyte sensing device,
or the injection means comprises a sharp longitudinal edge placed at the longitudinal side of the analyte sensing device.

According to another aspect there is provided a method for inserting an analyte sensing device into a subcutaneous tissue of a body by an inserter assembly, said inserter assembly comprising an insertion device comprising a cover with a first end and an opposite second end and a sensor carrier placed inside the cover comprising insertion means, said sensor carrier is moved from a first position to a second position by activating a first energy storage unit that is activated by a deployment device placed in the insertion device, the inserter assembly further comprising a sensor assembly comprising the analyte sensing device and inserted and placed into the tissue when the sensor carrier is in the second position; a sensor base placed at the second end of the cover comprising a first surface and an opposite second surface, the second surface comprising an adhesive; wherein the sensor assembly comprises a sensor module connected to the analyte sensing device, said sensor module moves together with the analyte sensing device, and in the second position of the sensor carrier the sensor module attaches to the first surface of the sensor base while the analyte sensing device is placed in the subcutaneous tissue when the sensor carrier reaches its second position.

Aptly a second energy storage unit is activated by activating means when the sensor carrier reaches the second position, and the sensor carrier is by the activating means moved to a third position different from the second position, and in a direction towards the first end of the cover.

### BRIEF DESCRIPTION OF THE DRAWINGS

FIG. 1 is a perspective view of an inserter assembly according to certain embodiments.

FIG. 2 is a view of the inside of the inserter assembly shown in FIG. 1.

FIG. 3 is a view of part of the inserter assembly shown in FIG. 1, including a piston, a sensor carrier, a sensor assembly, and a sensor base.

FIG. 4 illustrates certain components of the inserter assembly shown in FIG. 1, including a needle hub, a sensor carrier, a sensor assembly, and a sensor base.

FIG. 5A is an exploded view of a piston assembly.

FIG. 5B is a side and top perspective view of the sensor base comprising a sensor assembly and the sensor base is separated from a transmitter.

FIGS. 6A-C are sectional views of an inserter assembly according to certain embodiments, and illustrates energy storage units and the sensor carrier in a first position, a second position and a third position.

### DETAILED DESCRIPTION OF ILLUSTRATIVE EMBODIMENTS

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims. References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Items listed in the form of “A, B, and/or C” can also mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

With reference to FIG. 1, illustrated therein is an inserter assembly 1 according to the certain embodiments. The inserter assembly 1 is configured for inserting an analyte sensing device, such as a continuous glucose monitoring sensor, into a subcutaneous tissue of a body. The analyte sensing device could additionally or alternatively be a sensor for monitoring the inflammation level of the tissue, the pH value of the tissue, the number of the red and/or white corpuscles, and/or additional or alternative parameters. The inserter assembly 1 generally includes an insertion device 3 including a cover 4 with a first end 5 and an opposite second end 6. The inserter assembly 1 also includes a sensor base 12 placed at the second end 6 of the cover 4, the sensor base 12 comprising a base plate 13 with a first surface 14 and an opposite second surface 15. The second surface 15 comprises an adhesive adapted to adhere to a surface.

The sensor base 12 may be releasably connected, or unconnected to the second end 6 of the cover 4. The first surface 14 of the device 10 may have connection points to enable the cover 4 to attach to the sensor base 12.

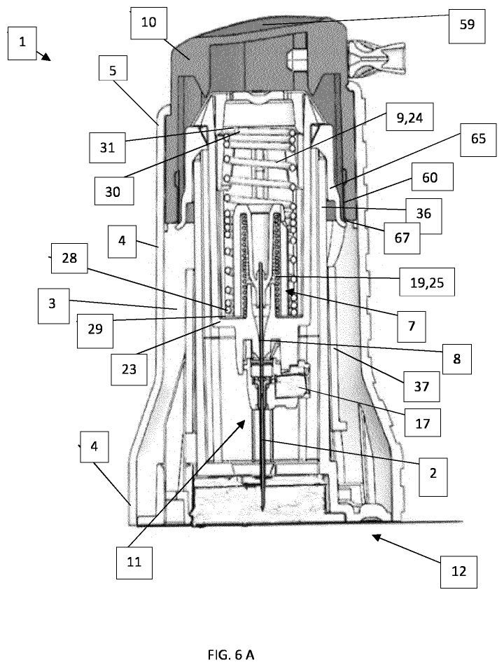

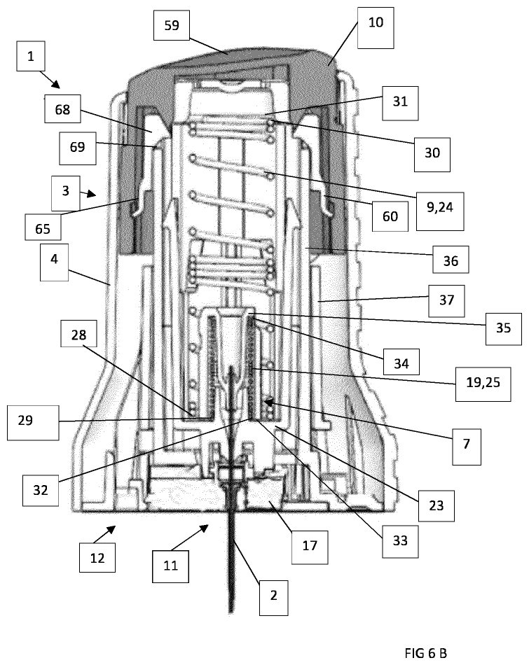

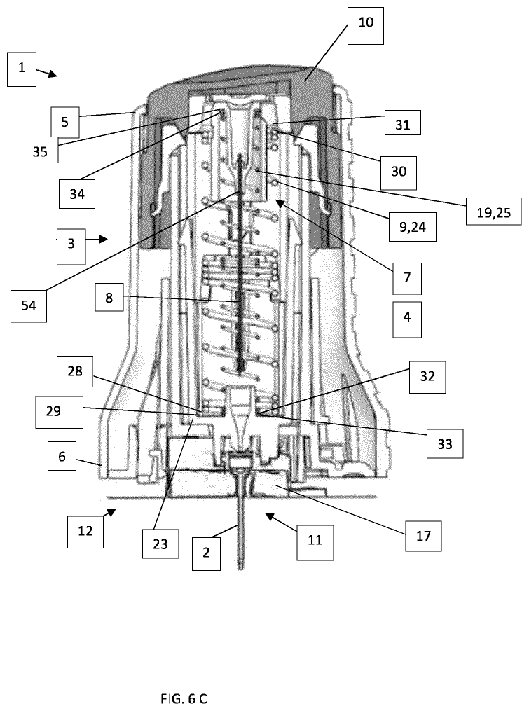

The illustrated inserter assembly 1 also includes a deployment device 10 placed in the first end 5 of the cover 6. While other forms of deployment device are contemplated, the illustrated deployment device 10 is provided in the form of a pressure button. The deployment device 10 activates a first energy-storage unit, and is to be described in further detail with reference to FIG. 6A-C. When a pressure is applied to the first surface 14 of the deployment device 10, the inserter assembly 1 is activated.

As described in further detail below, the cover 6 hides an inner housing 36, which houses a piston, a sensor carrier, a sensor assembly, the first storage unit, and a second energy storage unit.

In the illustrated form, a base lock 37 such as a slidable sleeve is placed around the inner housing 36, which includes releasable locking means adapted to lock the sensor base 12 to the insertion device 3. The locking means is configured to be released after reaching a second position by the action of the base lock 37, as described herein with reference to FIGS. 6A-C. The unlocking takes place by an axial movement of the base lock 37. The axial movement is executed by the piston, which includes pins that slide in longitudinally-placed recesses in the inner housing, thereby moving the base lock 37 in the direction towards the sensor base 12. The base lock 37 comprises flexible arms, the longitudinal axes of which may be parallel with the direction of movement. Further details regarding the function of the flexible arms are explained below with reference to FIG. 6A-C.

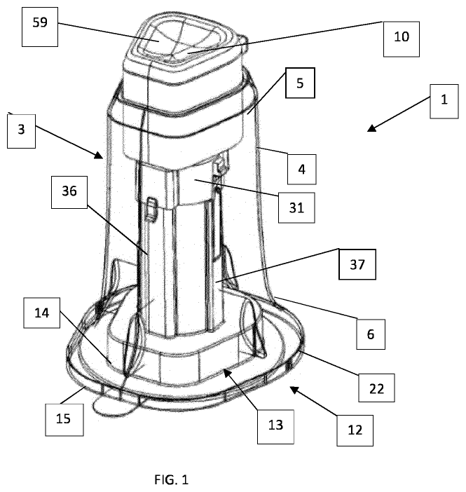

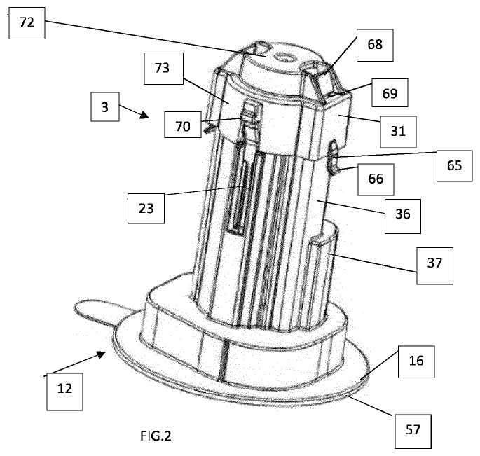

With additional reference to FIG. 2, illustrated therein is a view of the inside of the inserter assembly 1 shown in FIG. 1. In the illustration of FIG. 2, the cover 4 and the deployment assembly 10 have been removed to more clearly illustrate the remaining features. FIG. 2 further shows that the sensor base 12 at the second surface 15 of the base plate 13 is provided with an adhesive pad 16 covered by a removable release liner 57. A piston 23 is placed inside the inner housing 36 and comprises locking means 68 such as pins. The pins rest on an upper edge 69 of a release part 31, which surrounds the upper portion of the inner housing 36.

A cap includes a cap-surface 72 placed between the button and the inner housing, and a surrounding portion 73 encircling an upper portion of the outer surface of the inner housing 36. Flexible arms 65 are placed in the lower portion of the surrounding portion 73. The flexible arms 65 include retention units such as beards 66 resting toward a lower edge 67 of the deployment device 10 when the device is in a first position, as illustrated in FIG. 6A. By a pressure applied to the (top) first surface 59, the retention units 66 are moved in a mesial or radially-inward direction (towards the center of the device), whereby the upper edge 69 of the release part 31 is moved in opposite direction. As a result, the piston 23 is released, whereby a force stored in a first energy storing device is released to move the sensor carrier from the first position to the second position.

The illustrated piston further includes radial pointing pins 70 placed in the upper part of the piston 23. The pins 70 are slidably arranged in a recess of the inner housing 36. By the release of the piston 23, the pins are moved in the direction towards the sensor base 12. By this movement, the pins 70 push the base lock 37 such that the base lock 37 releases the locking mechanism between the sensor base 12 and the insertion device 3 as explained with reference to FIG. 6A-C.

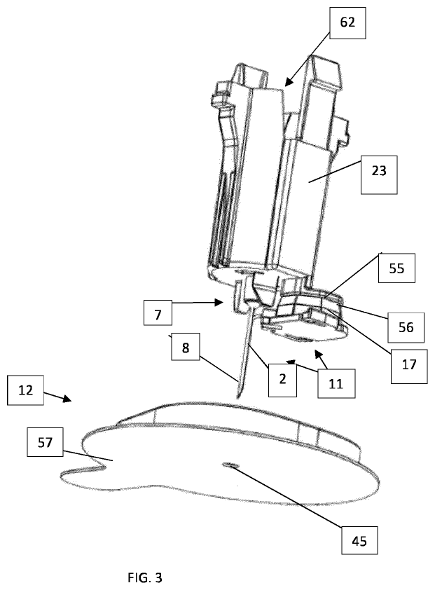

With additional reference to FIG. 3, the inserter assembly 1 further includes a piston 23, a sensor carrier 7, and a sensor assembly 11, one or more of which may be hidden inside the inner housing 36 in the illustrations of FIGS. 1 and 2. The sensor assembly 11 comprises an analyte sensing device, such as a continuous glucose monitoring sensor 2, and a sensor module 17. The sensor assembly 11 is attached to the sensor carrier 7, which includes injection means such as an introducer needle 8. The sensor carrier 7 comprises a cavity 62, which is open in the direction of the pressure button, and closed in the opposite direction by a needle hub that secures the introducer needle 8 to the sensor carrier 7. A second energy storage unit is housed in the cavity 62, and the continuous glucose monitoring sensor 2 is attached to the introducer needle 8.

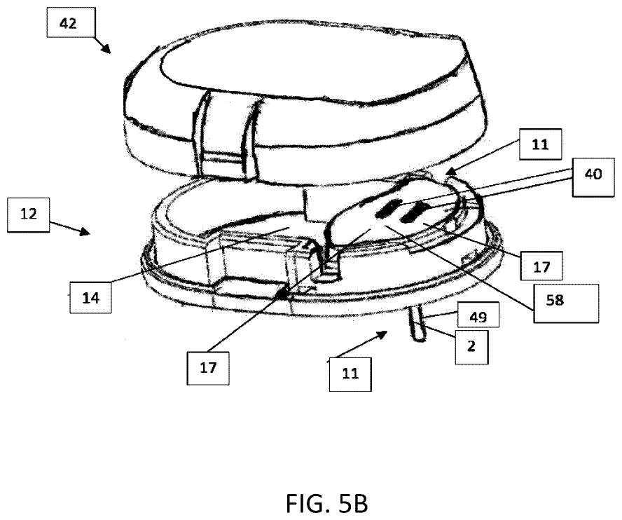

The piston 23 is the part that injects the continuous glucose monitoring sensor 2 into the tissue, and is activated by a spring as explained below. The introducer needle 8 is fastened to the lower part of the piston 23 in such a way that it follows the piston 23 in the direction towards the tissue, but is released from the piston 23 after the continuous glucose monitoring sensor 2 is introduced into the tissue and is moved in opposite direction away from the tissue. That is, (as described further below in relation to FIG. 5A) the introducer needle 8 is connected non-releasably to the needle hub 54, and that unit of hub and needle is located within the piston 23 and moves with the piston 23 towards the tissue. Then, as the needle hub meets the sensor base 12, there is a rotation of the needle hub that causes a second spring to be unlocked and the needle hub and introducer needle 8 to be forced back in the opposite direction.

A top surface 56 of the sensor module 17 abuts, and is releasably connected with a lower piston surface 55, such that the introducer needle 8, the continuous glucose monitoring sensor 2, and the sensor module 17 are moved synchronously during the injection of the sensor assembly 11. The top surface 56 of the sensor module and lower piston surface 55 may be connected by a high friction connection (e.g. by virtue of the surface roughness and material properties, such as a silicone membrane), or by a snap lock connection. This allows the respective surfaces to be held together before use, and then released when the sensor module 17 is fitted to the sensor base 12 after deployment. The continuous glucose monitoring sensor 2 may be injected through a through-going bore 45 in the sensor base 12 while the sensor module 17 is attached to the first surface 14 of the sensor base 12.

At the end of the insertion step, the sensor module gets released from the inserter device, by release of the high friction connection or snap lock, and attached to the sensor base.

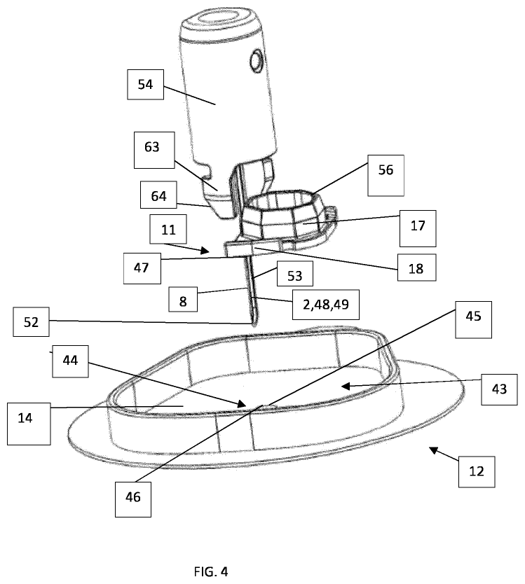

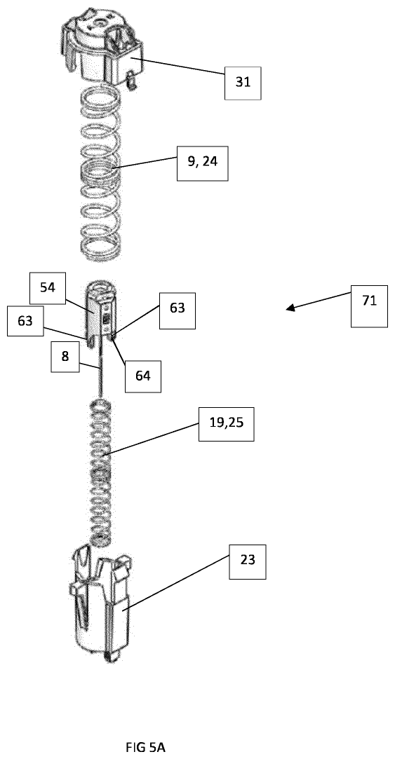

With additional reference to FIG. 4, illustrated therein are additional parts of the inserter assembly 1, including a needle hub 54, the sensor carrier 7, the sensor assembly 11, and the sensor base 12. The sensor assembly 11 comprises the sensor module 17 and the continuous glucose monitoring sensor 2. A lower part 48 of the continuous glucose sensor 2 is formed as an elongated pin-formed body 49. Further, the continuous glucose monitoring sensor 2 comprises an upper part 47, which is connected to the sensor module 17. In the illustrated example, the continuous glucose monitoring sensor 2 is not placed within the sensor module 17, but is instead placed laterally in relation to the sensor module 17 and connected to the sensor module 17 by the upper part 47 of the continuous glucose monitoring sensor 2. The connecting part may be bent to form an angle, such as an angle of around 90° (e.g., 85° to) 95°. The sensor 2 may be connected to the sensor module 17 by a wire, for example.

The continuous glucose monitoring sensor 2 is connected to the introducer needle 8, e.g. positioned within the needle 8 or at the tip of the needle 8. The introducer needle 8 of the sensor carrier 7 is, in sectional view, C-shaped, and comprises a sharp tip 52 and a lateral opening being the opening in the C-shape. This lateral opening comprises a sharp region 53 delimiting the opening. The introducer needle 8 is opposite the sharp tip 52 that is connected to the needle hub 54. The needle hub 54 is a part of the sensor carrier 7, and is moved downwards by the piston 23.

The first surface 14 of the sensor base 12 comprises a first area 43 adapted to receive the sensor module 17. The first surface 14 of the sensor base 12 further comprises a second area 44 that is separate from the first area 43, and which includes the through-going bore 45. The through-going bore 45 is adapted to receive a part of the continuous glucose monitoring sensor 2, which is injected through the bore 45 into the tissue. The continuous glucose monitoring sensor 2 is caught and secured to the sensor base 12 by third means 46. The third means 46 may be constructed as a conical part surrounding and keeping the continuous glucose monitoring sensor 2 in place by press fitting. The third means 46 may additionally or alternatively include flexible arms operable to catch grooves placed in a body surrounding the continuous glucose monitoring sensor 2.

The sensor module 17 may be fastened to the sensor base 12 in the first area 43 by taps or flexible arms 18, which are clipped into recesses constructed in the sensor base 12. The construction may also be the opposite, i.e. having taps or flexible arms constructed in the sensor base 12 that are clipped into recesses in the sensor module 17. Other fastening mechanisms may be used. For example, the sensor module 17 may be fastened by adhesive or by a push fit or wedge type of arrangement.

When the sensor assembly 11 is placed in the sensor base 12, the sensor carrier 7 is automatically withdrawn, thereby leaving the continuous glucose monitoring sensor 2 in the tissue. The sensor carrier 7 is withdrawn from the sensor assembly 11 by the activating of a second energy storage unit, which may be provided in the form of a coiled spring. The second energy storage unit is placed within the sensor carrier 7. A first energy storage unit, by which the introducer needle 8 carrying the sensor assembly 11 injects the continuous glucose monitoring sensor 2 into the tissue, is placed around the sensor carrier 7 but within the piston 23. Each of the energy storage units may, for example, be provided in the form of one or more preloaded springs.

FIG. 5A is an exploded view of a piston assembly 71 comprising a release part 31, a first energy storage unit 9, an introducer needle 8 fastened to a needle hub 54, a second energy storage unit 19, and the piston 23. The needle hub 54 is initially located within, and hooked into the piston 23 to move together. The lower part of the needle hub 54 comprises two elongated arms 63, each having a free end comprising a slanted edge 64. When the sensor carrier 7 is moved to the second position, the slanted edges 64 hit a corresponding surface of the sensor base 12. Hitting the sensor base 12 stops the travel of the piston 23. That is, the needle hub 54 (including needle 8), at the slanted edges 64 of the arms 63, contacts a correspondingly shaped portion of the sensor base 12, to cause rotation of the needle hub. So, by this interaction, the sensor carrier 7 (including the needle hub and insertion needle) is turned (rotated about 15-20 degrees) and released from the sensor assembly 11, and moved away inside the piston, thereby leaving the piston in its lower position. The needle hub rotation unhooks the hub from the piston. The second spring 19 is also unlocked and pushes the needle hub away from the skin.

FIG. 5B is perspective view of the sensor base 12 (including a sensor assembly 11), and a transmitter 42 separable from the sensor base 12. The insertion device 3 has been removed from the sensor base 12. The transmitter 42 is secured to the sensor base 12 by locking means such as snap means. The elongated pin-formed body 49 is injected into the tissue, while the upper part of the continuous glucose monitoring sensor 2 is secured to the sensor base and to the sensor module 17. The sensor module 17 is secured to the first surface 14 by first means 18 (see FIG. 4) such as flexible arms attaching to recesses placed in the first surface 14. The sensor module 17 has on its top-surface 58 (i.e., the surface pointing away from the first surface 14) electrical connectors 40. The electrical connectors 40 are connected to electrical connectors placed in the transmitter 42 when the transmitter 42 is attached and placed on the base part 12. The transmitter 42 comprises electrical circuitry for transmitting signals received from the sensor assembly 11 to a remote unit. The transmitter 42 may further comprise a power supply such as batteries.

FIGS. 6A-C are sectional views of an inserter assembly 1 according to certain embodiments, and illustrate the principle of energy storage units. FIG. 6A illustrates the sensor carrier 7 in a first position, FIG. 6B illustrates the sensor carrier 7 in a second position, and FIG. 6C illustrates the sensor carrier 7 in a third position. The first position (FIG. 6A) is before the continuous glucose monitoring sensor 2 is injected into the tissue and the device is in a secured and locked state. The second position (FIG. 6B) is when the continuous glucose monitoring sensor 2 is injected into the tissue but before the sensor carrier 7 is withdrawn. The third position (FIG. 6C) is when the sensor carrier 7 is withdrawn and the continuous glucose monitoring sensor 2 is placed in the tissue. The principle of the insertion device 3 is explained below with reference to FIGS. 6A-C. This principle is also the injection principle for the embodiment shown in the figures explained above.

The insertion device 3 comprises a first energy storage unit 9 and a second energy storage unit 19. In the illustrated form, the first energy storage unit 9 includes a first spring 24, and the second energy storage unit 19 includes a second spring 25. Each of the illustrated springs 24, 25 is a cylindrical coil spring, and a longitudinal central axis of the first spring 24 is coaxial with a longitudinal central axis of the second spring 25.

With the sensor carrier 7 in the first position, the first spring 24 is located within the hollow piston 23, which in turn is located inside the inner housing 36. The first spring 24 surrounds the sensor carrier 7. The second spring 25 is located inside the cavity of the hollow sensor carrier 7. The first spring 24 is preloaded with a first spring force F1 (e.g., about 12 N), and the second spring is preloaded with a second spring force F2. The first spring force F1 may be greater than the second spring force F2, and in certain embodiments may be at least twice the second spring force. For example, the first spring force F1 may be about 12 N, and the second spring force F2 may be about 5.5 N. Both preloaded springs may be locked before use.

In the first position, the sensor module 17 is spaced from (detached from) the first surface 14 of the sensor base 12.

When the deployment device 10 is activated by pressing at the first surface 59, the first spring 24 is released. At or around the same time, a flexible fastening means, which secures the piston 23 to the inner housing 36 in the first position, is deactivated by the movement of the deployment device 10 (e.g., the button) in a downward direction. The flexible fastening means may comprise the locking means 68 that are removed from the resting position towards the upper edge 69 of the release part 31 by the applied pressure. The release part 31 is placed immovably in relation to the inner housing 36.

The pressure button 10 comprises the first surface 59, to which the pressure is applied, and a second opposite surface region 60 adapted to lock the flexible arms 65 towards the outside of the piston 23. By the pressure applied to the first surface 59, the flexible arms 60 are moved in the mesial direction and thereby release the piston 23 as the beards/taps are moved in the opposite direction. The force F1 stored in the first energy storing device 9 is released, and moves the sensor carrier 7 from the first position to the second position.

The first spring 24 is in a compressed state when the sensor carrier 7 is in the first position. A first free end 28 of the first spring 24 is placed nearest the second end 6 of the cover 4, and presses toward an inside surface 29 of the piston 23. The second opposite placed free end 30 of the first spring 24 presses towards an opposite placed part that is an inner top surface of the release part 31.

The base lock 37 is positioned around a lower part of the inner housing 36, and is slightly slidable in the longitudinal direction towards the sensor base 12. In the illustrated form, the base lock 37 is a substantially cylindrical unit and comprises longitudinal flexible arms, the longitudinal axes of which may be parallel with the longitudinal axis of the insertion device 3. Each of the flexible arms comprises one or more beards 66. The insertion device and the sensor base are locked to each other by pins/tabs in one part interacting with recesses in the other part. When the base lock 37 is moved during the action from the first position to the second position and reaches the second position, the flexible arms and the beards thereon interact with the locking and removes the pins/tabs from the recesses. As a result, the inserter device and the sensor base are released from each other. This may be done after the sensor carrier has released the sensor assembly 11.

When the sensor carrier 7 reaches the second position, the sensor assembly 11 is released from the introducer needle 8 and the sensor carrier 7, as the sensor carrier 7 reaches a surface at the sensor base 12, thereby forcing the sensor carrier 7 to rotate. By the rotation caused by these activating means, the sensor assembly 11 is released, whereby the force F2 of the second spring 25 is released and the second spring 25 is expanded, thereby bringing the sensor carrier 7 into its third position. The second spring 25 is located inside the sensor carrier 7 in a cavity and a first free end 32 of the second spring 25 abuts against an internal base part 33 of the piston 23. The opposite second free end 34 of the second spring 25 abuts an upper surface 35 of the sensor carrier 7. When the second spring 25 is released as the sensor carrier is released from the sensor assembly, the sensor carrier 17 is moved in the direction opposite the injection direction, thereby reaching the third position.

The sensor base 12 is released from the insertion device 3 by the deactivating of the locking mechanism of the base lock 37. This is done by the flexible arms of the base lock 37, which are pressed to the sides and thereby miss the engagement to the recesses in the base part 12. When the locking mechanism is deactivated, the rotating movement of the sensor carrier 17 takes place. As a result of this rotational movement, locking means locking the sensor carrier to the piston are released, and the force of the second energy storage unit F2 is thereby released. As the base part 12 comprises an adhesive pad 16 placed at the second surface 15, the sensor base 12 is now fastened to the skin surface.

Preferably, the insertion needle of the sensor carrier is in sectional view C-shaped, and comprises a lateral opening delimited by sidewalls and a sharp tip. In certain embodiments, the lateral opening of the C-shape comprises a sharp region at both limiting sidewalls.

The insertion needle could also be a solid needle such as it is common known from the medical injection devices. In that case, the continuous glucose monitoring sensor may be formed hollow surrounding the insertion needle. The continuous glucose monitoring sensor could also be formed C-shape and partly surround the insertion needle. Additionally or alternatively, the sensor could be formed as an injection needle comprising a sharp tip and in some cases a sharp sidewall. Thereby it is not relevant with a separate insertion needle as the sensor itself ensures that the continuous glucose monitoring sensor is injected properly into the tissue.

With the above described arrangement, an inserter assembly is provided for inserting an analyte sensing device into the skin, moveable between a first position (before insertion), a second position (sensor injected) and a third position (sensor carrier withdrawn). The device is arranged such that deployment of the button 10 causes an axial movement of a piston 23 and sensor carrier towards a sensor base 12. A sensor 2 is injected into tissue and a sensor module 17 is attached to the sensor base 12. The sensor carrier is retracted, and the insertion device may be removed to leave the sensor base 12 attached to the skin.

A transmitter 42 may be connected on the sensor base 12.

A sensor assembly engages with a sensor base only as the analyte sensor is implanted into the skin, i.e. only as the sensor module moves to the second position.

A transmitter may be connected to a sensor base (including sensor assembly) after the sensor assembly has been attached to the skin. In this way, the transmitter does not need to be attached to the skin as part of the sensor assembly. This gives more design freedom to the inserter assembly, enabling a simple construction. In addition, the transmitter may be interchanged if required, without changing the whole sensor assembly.

Furthermore, the construction of the inserter assembly enables a compact arrangement of the features to enable injection of the sensor 2 into a subcutaneous tissue.

In order to secure the placement of the sensor module into subcutaneous tissue, with the above arrangement, the configuration and the timing of the release and retraction of the insertion needle and rotation do not damage the sensor.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. An inserter assembly for inserting an analyte sensing device into subcutaneous tissue of a body, the inserter assembly comprising:
an insertion device comprising a cover with a first end and an opposite second end; and
a sensor carrier placed inside the cover, the sensor carrier comprising an injection means, wherein the sensor carrier is operable to be moved from a first position to a second position by activating a first energy storage unit, wherein the first energy storage unit is activated by a deployment device placed in the insertion device;
a sensor assembly comprising an analyte sensing device, wherein the sensor assembly is adapted to be inserted and placed into the subcutaneous tissue when the sensor carrier is in the second position;
a sensor base placed at the second end of the cover, the sensor base comprising a first surface and an opposite second surface, the second surface comprising an adhesive;
wherein the sensor assembly comprises a sensor module connected to the analyte sensing device, wherein the sensor module is adapted to be moved together with the analyte sensing device by a piston;
wherein, with the sensor carrier in the second position, the sensor module is adapted to be attached to the first surface of the sensor base; and
wherein the analyte sensing device is placed in the subcutaneous tissue when the sensor carrier reaches its second position.

2. An inserter assembly according to claim 1, wherein the inserter assembly further comprises a second energy storage unit;
wherein the second energy storage unit is activated by activating means when the sensor carrier reaches its second position;
wherein the sensor carrier is adapted to be moved, by the activating means, to a third position different from the second position, and in a direction towards the first end of the cover.

3. An inserter assembly according to claim 2, wherein the first energy storage unit comprises a first spring configured to bring the sensor carrier from the first position to the second position; and
wherein the second energy storage unit comprises a second spring adapted to transport the sensor carrier to the third position; and
wherein a longitudinal central axis of the first spring is coincident with a longitudinal central axis of the second spring.

4. An inserter assembly according to claim 3, wherein the first spring is a coil spring placed inside the piston and surrounding the sensor carrier;
wherein, when the sensor carrier is in the first position, the first spring is compressed, with a first free end placed nearest the second end of the cover and pressing towards an inside surface of the piston, and the second opposite free end of the first spring is pressed towards a part of the inserter assembly placed opposite the inside surface of the piston.

5. An inserter assembly according to claim 3, wherein the second spring is a coil spring and placed inside the sensor carrier in a cavity;
wherein a first free end of the second spring abuts an internal base part of the piston; and
wherein an opposite second free end of the second spring abuts an internal upper surface of the sensor carrier.

6. An inserter assembly according to claim 1, wherein the sensor base is adapted to cover a passage encircled by the second end of the cover, whereby the sensor carrier and the sensor assembly are enclosed in a cavity formed by the cover and the sensor base; and
wherein the inserter assembly comprises means adapted to attach the sensor base to the insertion device.

7. An inserter assembly according to claim 1, wherein an inner housing encircles the piston, and a base lock is placed around the inner housing;
wherein the base lock is slidable in a longitudinal direction of the inserter assembly;
wherein the inner housing comprises releasable locking means adapted to lock the sensor base to the insertion device when the sensor carrier is in the first position; and
wherein the locking means is adapted to be released by the base lock when the sensor carrier is in the second position to thereby release the sensor base from the insertion device.

8. An inserter assembly according to claim 7, wherein the base lock comprises a sleeve surrounding a portion of the inner housing; and
wherein the inserter assembly further comprises flexible arms, longitudinal axes of which are parallel with a longitudinal axis of the inserter assembly.

9. An inserter assembly according to claim 1, wherein, upon reaching the second position, the sensor base and analyte sensing device are detachable from the remaining parts of the inserter assembly.

10. An inserter assembly according to claim 1, wherein the analyte sensing device is connected to the sensor module by a wire.

11. An inserter assembly according to claim 1, wherein the sensor module comprises electrical connectors for connecting to an electrical circuitry and for transmitting signals received from the analyte sensing device to a transmitter.

12. An inserter assembly according to claim 11, further comprising the transmitter, and wherein the transmitter comprises electrical circuitry for transmitting signals received from the sensor assembly to a remote unit; and
wherein the transmitter further comprises a power supply.

13. An inserter assembly according to claim 1, wherein the first surface of the sensor base comprises a first area adapted to receive the sensor module;
wherein the sensor base further comprises a second area separated from the first area, the second area comprising a through-going bore in said sensor base;
wherein said through-going bore is adapted to receive a part of the analyte sensing device and to secure an upper part of the analyte sensing device to the sensor base.

14. An inserter assembly according to claim 9, wherein the second area further is adapted to fasten an upper part of the analyte sensing device to the sensor base while a lower part of the analyte sensing device comprises an elongated body that is injected through the bore and is placed in the subcutaneous tissue when the sensor carrier reaches its second position.

15. An inserter assembly according to claim 1, wherein the injection means comprises an insertion needle, which in sectional view is C-shaped and comprises a sharp tip and a lateral opening delimited by sidewalls, and wherein the lateral opening comprises a sharp region at each of the sidewalls.

16. An inserter assembly according to claim 1, wherein the sensor carrier comprises a needle hub attaching the insertion needle; and
wherein the needle hub is configured to be moved by the piston from the first to the second position.

17. An inserter assembly according to claim 16, wherein the sensor carrier comprises a hollow part in which the needle hub is placed; and
wherein the second energy storage unit is placed in a circular cavity arranged between the outside of the needle hub and the inside of the hollow part when the sensor carrier is in the first position and the second position.

18. An inserter assembly according to claim 1, wherein the piston comprises a lower piston surface part adapted to abut against a top surface of the sensor module, wherein said piston surface and the injection needle are moved synchronously from the first position to the second position, and the injection needle and the lower piston surface part are placed in a distance from each other.

19. An inserter assembly according to claim 1, further comprising flexible elongated retention arms by which the sensor module is operable to be attached to the first surface of the sensor base, the retention arms comprising retention means adapted to be locked into recesses.

20. An inserter assembly according to claim 2, wherein the activating means is configured to cause a rotating movement of the sensor carrier whereby locking of the sensor carrier to the piston is released, and a force of the second energy storage unit is released thereby moving the sensor carrier from the second position to the third position.

21. An inserter assembly according to claim 20, wherein the rotation movement is caused by connection of a slanted surface of the sensor carrier moving against a corresponding surface of the sensor base.

22. An inserter assembly according to claim 1, wherein the inserter assembly comprises a release part comprising a cap with a cap surface placed between the deployment device and the inner housing, and a surrounding cap-portion encircling an upper part of the outer surface of the inner housing, and further comprising flexible arms locking the deployment device when the sensor carrier is in the first position.

23. An inserter assembly according to claim 22, wherein the deployment device comprises a pressure unit comprising a first surface to which pressure is applied and a second opposite surface region adapted to lock flexible arms placed in the release part towards a lower edge of the deployment device;
wherein the flexible arms comprise retention units; and
wherein by a pressure applied to the first surface the retention units are moved to thereby release the piston, whereby a force stored in the first energy storing device is released and moves the sensor carrier from the first position to the second position.

24. An inserter assembly according to claim 23, wherein the piston comprises locking means resting on an upper edge of the release part;
wherein said locking means are adapted to be released from the release part by a lateral movement of the upper part, which takes place when the retention units are moved.

25. An inserter assembly according to claim 2, wherein the first energy storage unit is preloaded with a first expansion force;
wherein and the second energy storage unit is preloaded with a second expansion force; and
wherein the first expansion force is larger than the second expansion force.

26. An inserter assembly according to claim 22, further comprising a piston assembly comprising the release part, a first energy storing device, an insertion needle fastened to a needle hub, a second energy storing device, and the piston.

27. An inserter assembly for inserting an analyte sensing device into subcutaneous tissue of a body, said inserter assembly comprising:
an insertion device comprising a cover with a first end and an opposite second end and a sensor carrier placed inside the cover, wherein said sensor carrier is moved from a first position to a second position by activating a first energy storage unit, wherein the first energy storage unit is activated by a deployment device placed in the insertion device;
a sensor assembly comprising:
the analyte sensing device, wherein the sensor assembly adapted to be inserted and placed into the tissue when the sensor carrier is in the second position;
a sensor base placed at the second end of the cover, the sensor base comprising a first surface and an opposite second surface, the second surface comprising an adhesive; and
a sensor module connected to the analyte sensing device, said sensor module is adapted to be moved together with the analyte sensing device by a piston;
wherein, with the sensor carrier in the second position, the sensor module is adapted to be attached to the first surface of the sensor base; and
wherein the analyte sensing device is by injection means placed in the subcutaneous tissue when the sensor carrier reaches its second position.

28. An inserter assembly according to claim 27, wherein the injection means comprises a sharp tip of the analyte sensing device,
or wherein the injection means comprises a sharp longitudinal edge placed at the longitudinal side of the analyte sensing device.

29. A method for inserting an analyte sensing device into a subcutaneous tissue of a body by an inserter assembly, said inserter assembly comprising an insertion device comprising a cover with a first end and an opposite second end and a sensor carrier placed inside the cover comprising insertion means, said sensor carrier is moved from a first position to a second position by activating a first energy storage unit that is activated by a deployment device placed in the insertion device, the inserter assembly further comprising a sensor assembly comprising the analyte sensing device and inserted and placed into the tissue when the sensor carrier is in the second position; a sensor base placed at the second end of the cover comprising a first surface and an opposite second surface, the second surface comprising an adhesive; wherein the sensor assembly comprises a sensor module connected to the analyte sensing device, said sensor module moves together with the analyte sensing device, and in the second position of the sensor carrier the sensor module attaches to the first surface of the sensor base while the analyte sensing device is placed in the subcutaneous tissue when the sensor carrier reaches its second position.

30. A method according to claim 29, wherein a second energy storage unit is activated by activating means when the sensor carrier reaches the second position, and the sensor carrier is by the activating means moved to a third position different from the second position, and in a direction towards the first end of the cover.
